(19) United States
(12) Patent Application Publication
(10) Pub. No.: US 2005/0256400 A1
(43) Pub. Date: Nov. 17, 2005

(54) Method to identify arterial and venous vessels

(76) Inventors: Bhargav Raman, Cupertino, CA (US); Raghav Raman, Cupertino, CA (US); Sandy A. Napel, Menlo Park, CA (US); Geoffrey D. Rubin, Woodside, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/003,584

(22) Filed: Dec. 2, 2004

Related U.S. Application Data
(60) Provisional application No. 60/526,560, filed on Dec. 3, 2003.

(51) Int. Cl. A61B 5/05

(52) U.S. Cl. 600/425

(57) ABSTRACT

A method for identifying a arteries and veins in a medical image is provided. A start point and endpoints of branches of a segmented tubular tree are identified. Distance maps for each of the endpoints relative to the startpoint are created. Then voxels in between the furthest of the endpoints and the startpoint are identified. This last step is iterated for the subsequent furthest of the endpoints. For each set of identified voxels parameters are identified. Examples of such parameters are cross sectional areas of the branches. The parameters for at least one each set of identified voxels are then used to anatomically label branches the segmented tubular tree, optionally with position information obtained from the image.

### CROSS-REFERENCE TO RELATED APPLICATIONS

This application is cross-referenced to and claims priority from U.S. Provisional Application 60/526,560 filed Dec. 2, 2003. All these applications are hereby incorporated by reference.

### STATEMENT REGARDING FEDERALLY SPONSORED RESEARCH OR DEVELOPMENT

The present invention was supported in part by grant numbers 5R01HL58915 and 1R01HL67194 both from the National Institutes of Health (NIH). The U.S. Government has certain rights in the invention.

### FIELD OF THE INVENTION

The present invention relates generally to medical imaging. More particularly, the present invention relates to a method for identifying vessels.

### BACKGROUND

Vessel structures in the body are routinely evaluated for vascular disease using imaging modalities such as Computed Tomographic Angiography (CTA) or Magnetic Resonance Angiography. The images thus obtained constitute a volumetric dataset which contains the vascular tree of the human body. The exact locations, sizes and lengths of the tree vary widely between patients. To review the vessels for disease, the clinician routinely has to identify the anatomic labels, origins, course and extent of these vessels. Currently the art lacks methods to ease the task of identifying the anatomic labels of vessels. Accordingly, it would be considered an advance in the art to identify the anatomic labels of vessels in radiological images.

### SUMMARY OF THE INVENTION

The present invention is a method for identifying a tubular tree in a medical image. A start point and endpoints of branches of a segmented tubular tree are identified. Distance maps for each of the endpoints relative to the startpoint are created. Then voxels in between the furthest of the endpoints and the startpoint are identified. This last step is iterated for the subsequent furthest of the endpoints. For each set of identified voxels parameters are identified. Examples of such parameters are cross sectional areas of the branches. The parameters for at least one each set of identified voxels are then used to anatomically label branches of the segmented tubular tree, optionally with position information obtained from the image. In one embodiment, irregularities, discontinuities or changes in the parameters are used to anatomically label branches of the segmented tubular tree. Examples of a tubular tree are e.g. arterial or venous vessels.

### BRIEF DESCRIPTION OF THE FIGURES

The objectives and advantages of the present invention will be understood by reading the following detailed description in conjunction with the drawings.

FIG. 1 shows an example of finding a start-point of a segmented vessel tree according to the present invention.

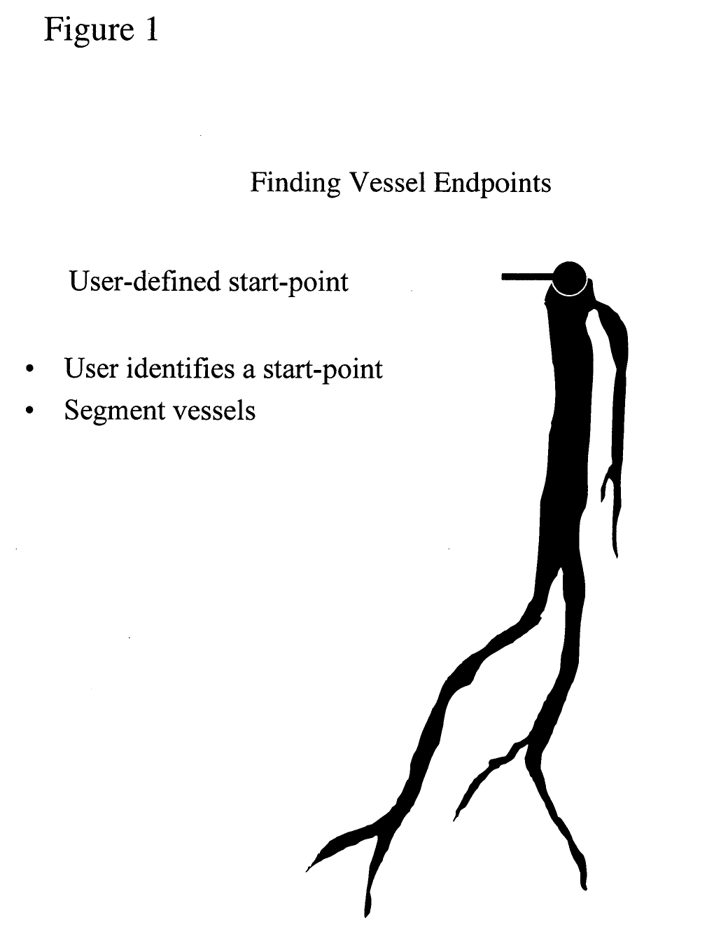

FIG. 2 shows an example of creating a distance map of the segmented vessel tree according to the present invention. The distance map is an enumeration of the distance of each voxel in the segmentation to the start-point.

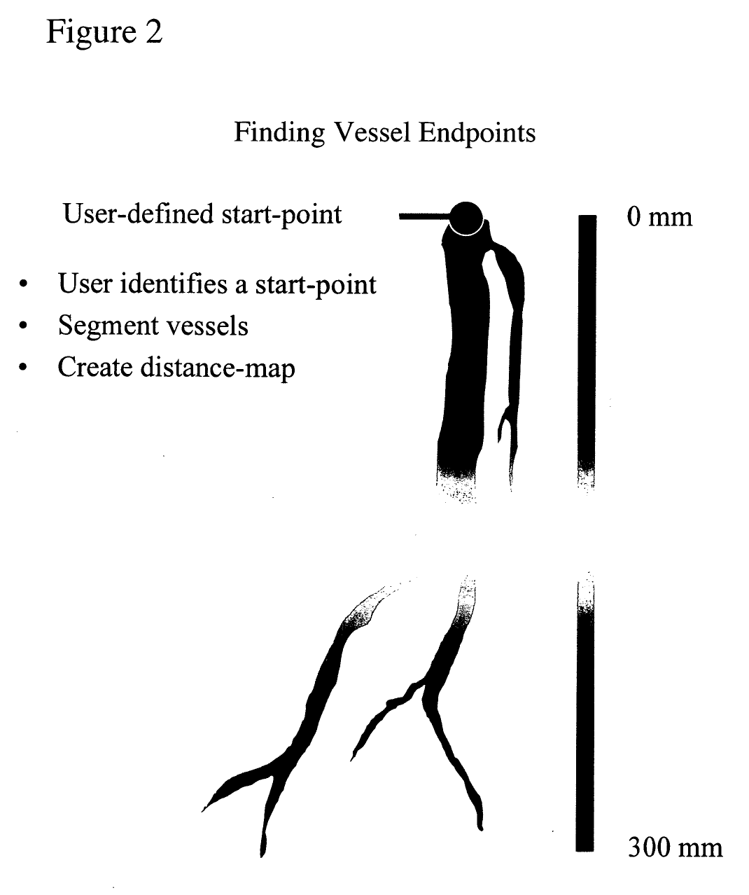

FIG. 3 shows an example finding the first and furthest endpoint as the point, which has the highest enumerated distance according to the present invention

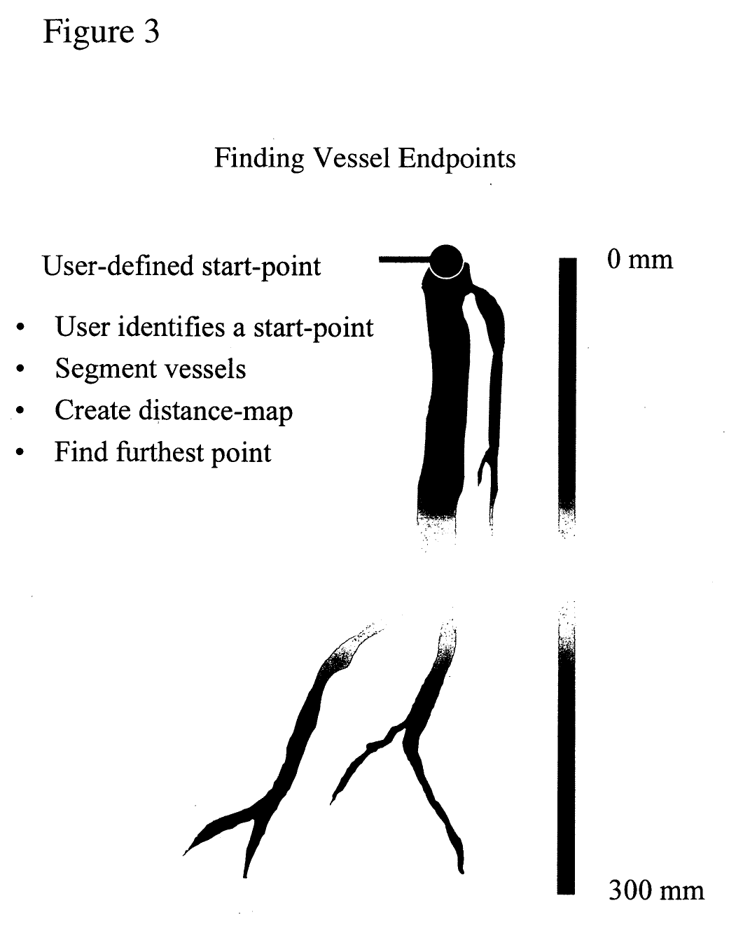

FIG. 4 shows an example of masking the voxels that are considered to belong to the branch of the first endpoint according to the present invention. This is done by iteratively selecting all voxels that have a distance less than the endpoint without selecting the voxels with a distance higher than the selected voxels.

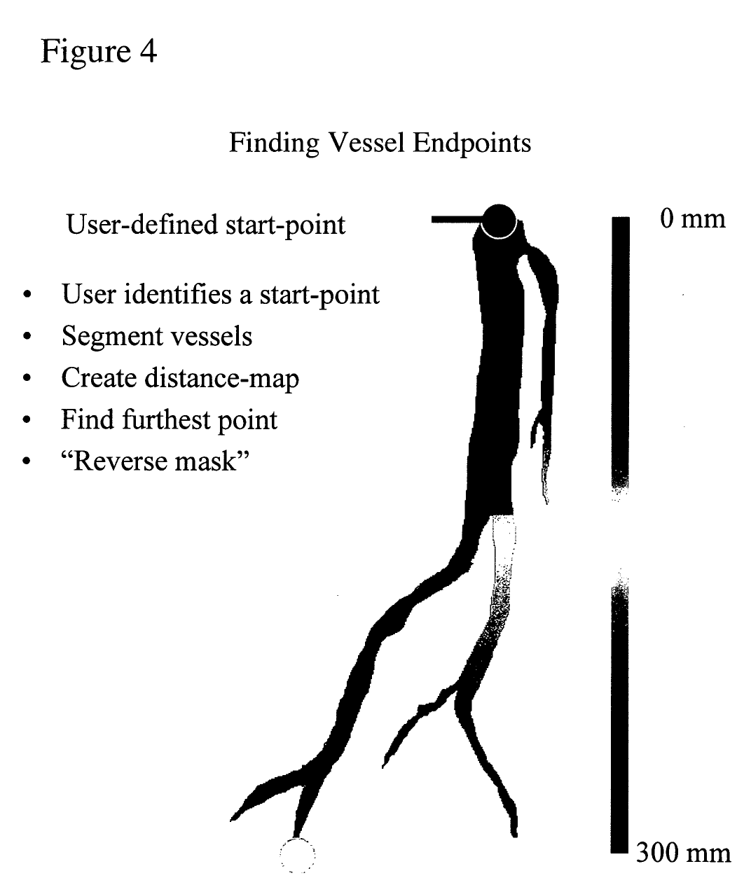

FIG. 5 shows an example of finding the next furthest endpoint.

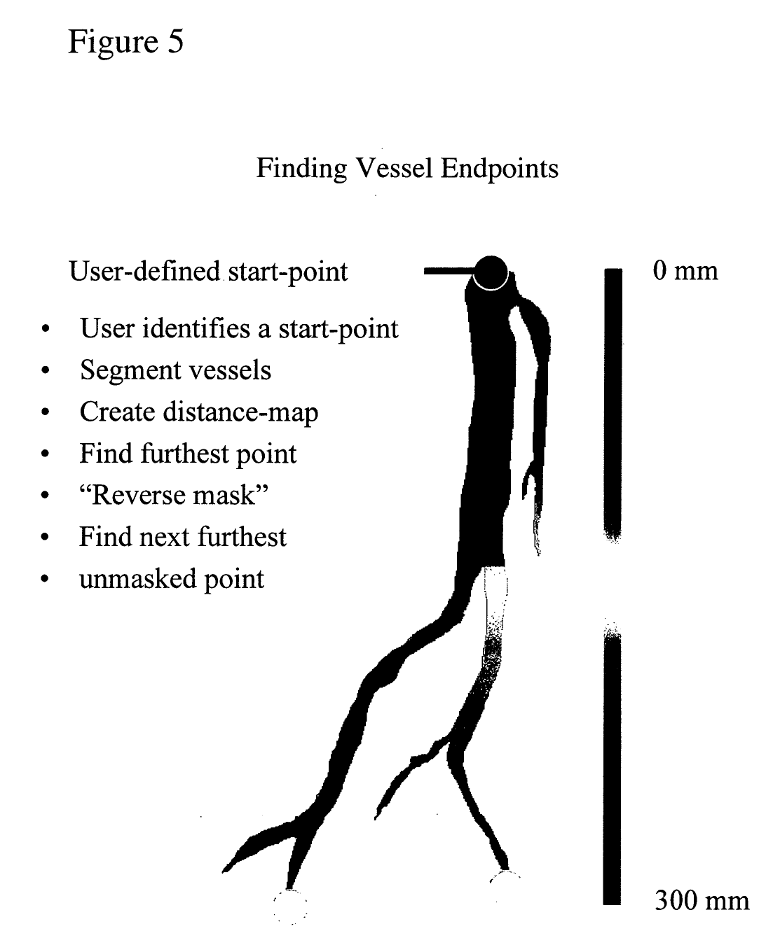

FIG. 6 shows an example of repeating the steps in FIG. 4 for the next endpoint found in FIG. 5 according to the present invention.

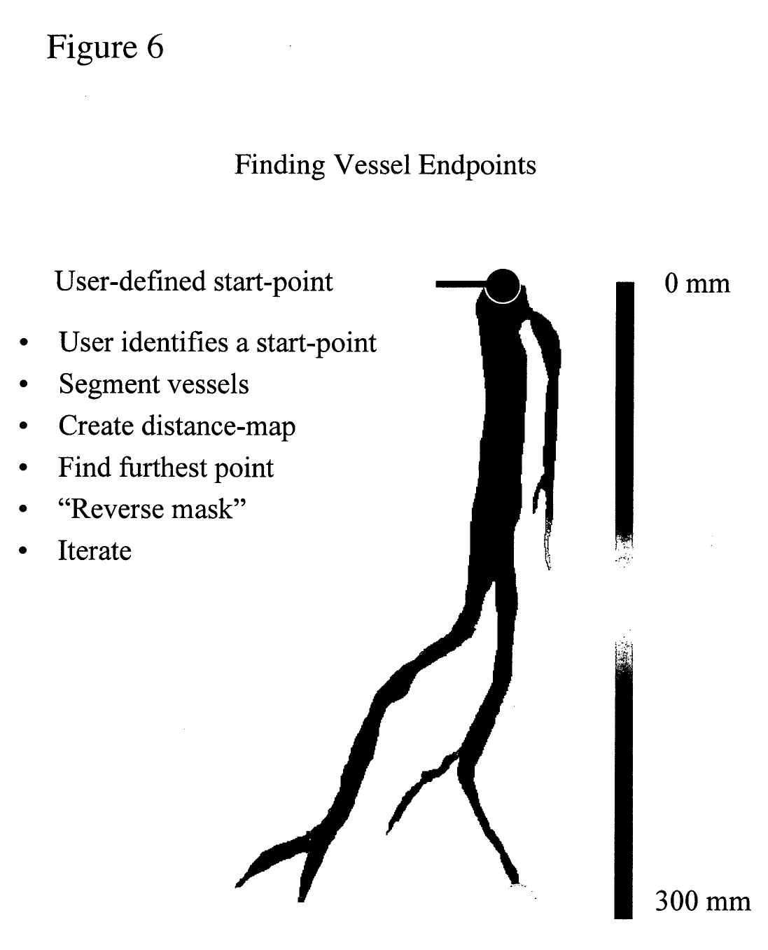

FIG. 7 shows an example of iterating the steps of FIGS. 5-6 until there are no more voxels to be selected.

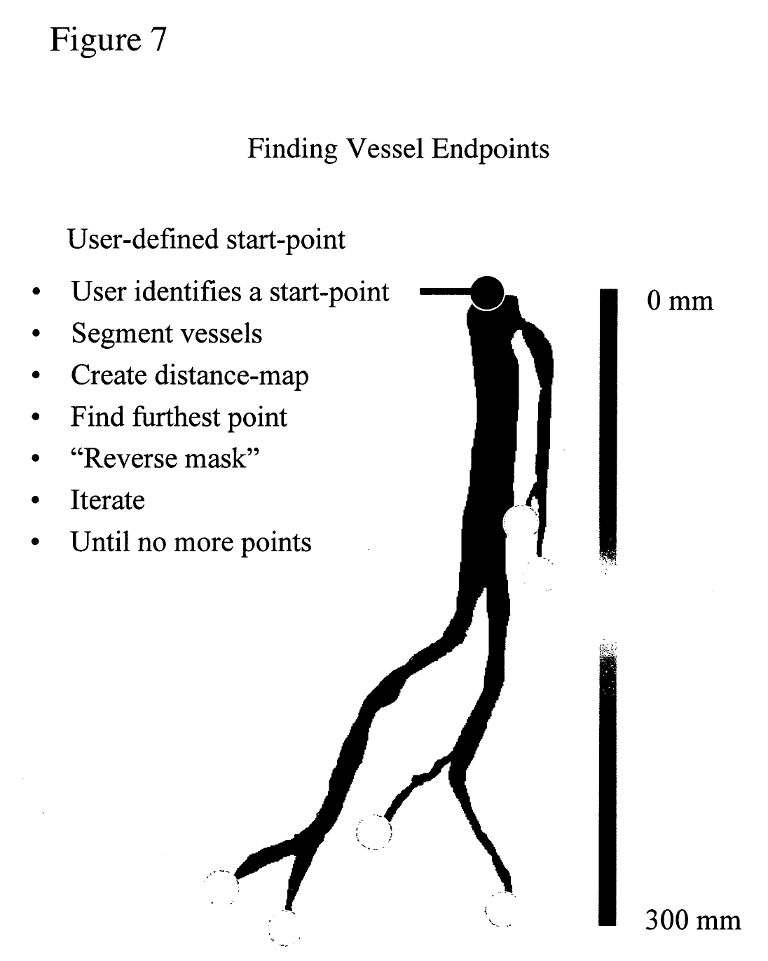

FIG. 8 shows an example of finding the centerline paths for each identified branch. This step of finding the centerline is optional.

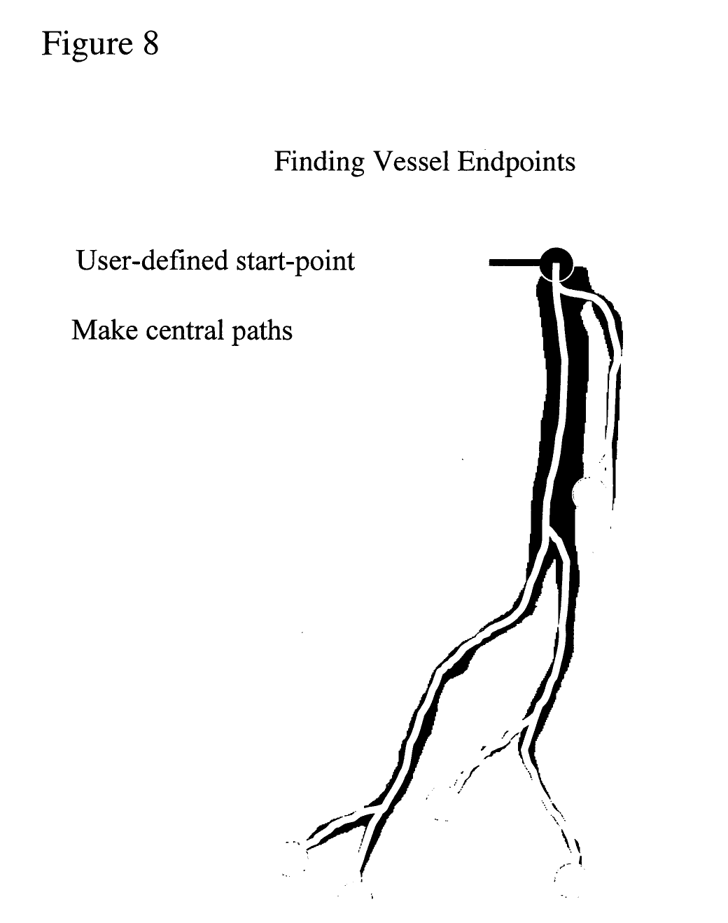

FIG. 9 shows an example anatomical labeling using the cross sectional area profile along the course of each identified branch. The labeling is based on using the disruptive or discontinuities in the cross sectional area profile according to the present invention.

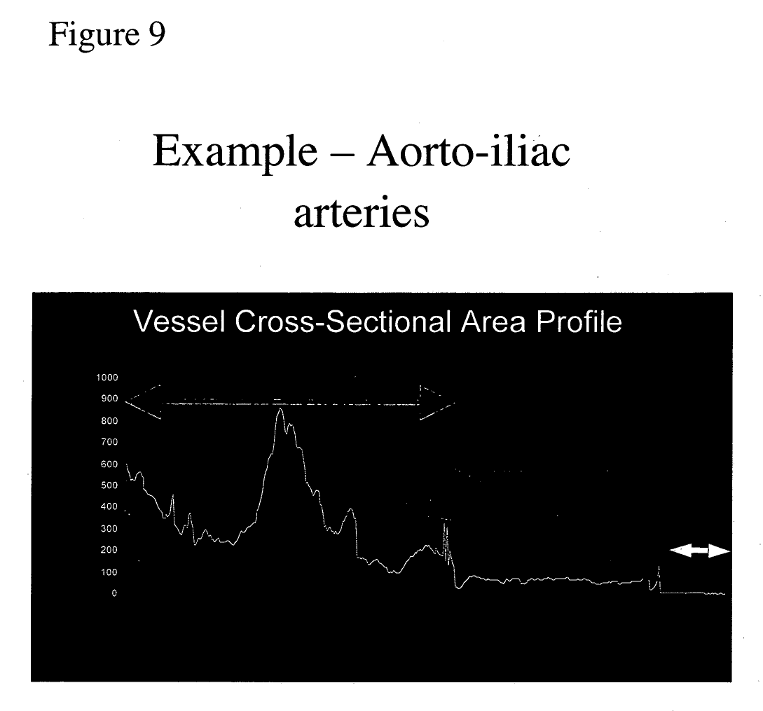

### DETAILED DESCRIPTION OF THE INVENTION

We have developed a method for automatically identifying the location and course of the vascular tree, given only one user-defined point in the root or parent vessel for the vascular tree. Our method also then uses the relatively invariant parameters (vessel cross sectional profile and vessel cross sectional area profile discontinuities, branching patterns, branch directions and laterality) of the human vascular tree to apply appropriate anatomic labels to the branches of the vascular tree.

The methods uses one (manually or automically) entered point in a vessel, e.g. the aorta, and patient orientation from the image headers to obtain position information for anatomic labeling. The method then creates a segmentation of the vessel tree. This is done by using an adaptive threshold and the startpoint as the seed point. The segmentation thus obtained represents the vascular tree in its entirety. A standard distancemap is then calculated. This distancemap enumerates the distance of each voxel in the segmentation to the startpoint. The first step in automatically identifying the endpoints of the branches of this vascular tree is to identify the voxel that has the furthest enumerated distance in the distancemap from the startpoint. This point is designated as the first branch endpoint that is identified. From this branch endpoint, an iterative reverse-masking procedure is applied as follows to select all voxels that are considered to be along the course of the first branch. Firstly, all voxels adjacent to the first branch endpoint that have a distance less than the branch endpoint are selected. For each of these newly selected voxels, all unselected voxels that have a smaller distance than the current voxel are selected. This process therefore selects only voxels that are closer to the startpoint. This process is iterated until no more unselected voxels remain that are closer to the startpoint. Because only voxels closer to the startpoint than the current voxel are selected at any time, voxels that belong to other branches are never selected.

Following this step, the second branch endpoint is selected as the unselected voxel with the highest enumerated distance from the startpoint. The iterative reverse-masking procedure described above is then reapplied to select the voxels that are considered to belong to this second branch endpoint. This step of selecting endpoints followed by reverse masking is then repeatedly applied to select the subsequent endpoints, until no more unselected voxels remain. In this way, every endpoint of the vascular tree is identified. In an optional embodiment to the algorithm, a further filtering step can be then applied to delete branches that contain less than a certain number of voxels. This would allow the automated deletion of minor branches that have a volume less than what is considered clinically significant.

Once the clinically significant endpoints have been identified, the method optionally generates branching central paths from the startpoint to the endpoints. This can be done with any median path generation algorithm.

Then the method applies the appropriate anatomic labels to each of the branches identified. This is accomplished as follows: Firstly, the method has to identify the branchpoints of the vessels. A cross-sectional area profile from the startpoint to each endpoint is calculated and plotted. The method then identifies sharp discontinuities or irregularities in the cross sectional area profile that correspond to the wide fluctuations of cross sectional area that are seen at branchpoints. Then, the change in cross sectional area proximal to and distal to these sharp irregularities is quantified and compared to a heuristic anatomical model to identify the anatomic label to be applied to the vessels proximal and distal to the identified branchpoints. This anatomical model also takes into account the vector direction of the branch proximal and distal to the identified branchpoints. In addition, coordinate information from the image or position information could be used to e.g. identify lateral/medial, anterior/posterior and/or superior/inferior labels of branches

We validated our algorithm using CTAs from 7 consecutive patients with aortic aneurysms. Positive vessel ID and the number of vessels missed were scored by an experienced 3D technologist, and 4 radiologists independently identified both patent and occluded segments. The error in localization of origins, and the time required were quantified and compared to manual identification. In addition, the mean identified length of vessels and the mean diameter of the most distal vessel segments identified was measured.

Arteries that could be identified by the method include the aorta, celiac trunk, hepatic, splenic, superior mesenteric artery (sma), pulmonary artety and veins, renals and common, internal and external iliac arteries and their major branches. It also identifies several other more minor arteries such as the ima, umbilical and gastrointestinal arteries, and identifies the existence of multiple instances of the same arteries, for example, usually originate from the celiac, but also sometimes originate from the sma. However, the present invention could not only be useful for vessels but to tubular tree structures in general not limiting to blood vessels such as lymphatic trees.

The present method and system can be integrated into automated post-processing and reporting systems which then greatly reduces operator time and costs.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations and other variations are considered to be within the scope and spirit of the present invention as defined by the following claims and their legal equivalents.

1. A method for identifying a tubular tree in a medical image, comprising the steps of:
(a) identifying a start point and endpoints of branches of a segmented tubular tree;
(b) create a distance map for each of said endpoints relative to said startpoint;
(c) identify voxels in between the furthest of said endpoints and said startpoint that are closer to said startpoint, but not further than the current voxel or voxels to said startpoint;
(d) iterate said step (c) for the subsequent furthest of said endpoints;
(e) determine parameters for each set of identified voxels; and
(f) using said parameters for at least one each set of identified voxels to anatomically label branches of said segmented tubular tree.

2. The method as set forth in claim 1, wherein said parameters are cross sectional areas of said branches.

3. The method as set forth in claim 1, further comprising the step of identifying irregularities, discontinuities, or changes in said parameters to anatomically label branches of said segmented tubular tree.

4. The method as set forth in claim 1, wherein said tubular tree is an arterial and a venous structure each with branches.
